(19) United States
(12) Patent Application Publication
(10) Pub. No.: US 2007/0110823 A1
(43) Pub. Date: May 17, 2007

(54) Sintered bioactive ceramic composite implant and preparation thereof

(76) Inventors: Young Min Kong, Daejeon-city (KR); Eungje Lee, Seoul (KR); Jongsik Choi, Daejeon-city (KR); Hyoun Ee Kim, Seoul (KR)

Correspondence Address: McKenna Long & Aldridge, 1900 K Street N W, Washington, DC, 20006, US

(21) Appl. No.: 10/563,253

(22) PCT Filed: Oct. 11, 2005
(86) PCT No.: PCT/KR05/03363

§ 371 Date: Jan. 4, 2006

(30) Foreign Application Priority Data
Oct. 11, 2004 (KR) 10-2004-0081110
Oct. 10, 2005 (KR) 10-2005-0094798

(51) Int. Cl. A61K 33/42; A61K 33/24; B28B 5/00

(52) U.S. Cl. 424/602; 977/906; 424/617; 264/603

(57) ABSTRACT

A sintered bioactive ceramic composite for implant having high strength and superior bioactivity and a method of preparing the same are provided. The composite includes a zirconia-alumina nano-composite-powder and an apatite-related compound and is useful for a load-bearing implant.

### BACKGROUND OF THE INVENTION

1. Field of the Invention

The present invention relates to a sintered ceramic composite and a method of preparing the same. More particularly, the present invention relates to a sintered bioactive ceramic composite for implant having bioactivity similar to an apatite-related compound and high strength, and a method of preparing the same.

2. Description of the Related Art

Apatite-related compounds, which are calcium phosphate-based compounds, have crystallographic and chemical characteristics similar to various hard tissues such as bones and teeth of vertebrata, and thus strongly bind to biotissues when they are transplanted in a body. Hydroxyapatite (HA, Ca₁₀(PO₄)₆(OH)₂) is known as a representative apatite. There have been efforts to replace impaired teeth and bones with hydroxyapatite by using its bioactivity. However, since mechanical properties such as strength and fracture toughness of hydroxyapatite are poor, its use is limited to non load-bearing part such as auditory ossicle. To use hydroxyapatite having good bioactivity as a load-bearing bioactive ceramic implant, various composites thereof were proposed.

Hydroxyapatite composites may be divided into a macrocomposite for improving biocompatibility of metal implants by applying a hydroxyapatite coating layer to the surface of a metal base and a microcomposite for improving physical properties of a hydroxyapatite matrix phase by adding a secondary phase having high strength to the hydroxyapatite matrix phase. However, both composites still have problems when applied to the load-bearing implant.

In the case of the macrocomposite, the coating layer is peeled off due to a difference in physical property between the metal base and the hydroxyapatite coating layer, and the heat treatment in a coating process and a subsequent process results in a change in the physical property of metal.

Meanwhile, the microcomposite is decomposed due to contact of the bioactive hydroxyapatite matrix phase and the bioinert secondary phase material during sintering the composite. As a result, most of the bioactive hydroxyapatite matrix phase is converted into a bioresorbable tricalcium phosphate (TCP, Ca₃(PO₄)₂), resulting in a reduction in bioactivity of the hydroxyapatite composite and a significant reduction in mechanical properties due to a change (decrease) in physical properties of the secondary phase. To avoid these problems, Korean Patent No. 294008 discloses the formation of a barrier layer on the surface of the secondary phase to prevent contact of the secondary phase and the HA matrix phase, which inhibits the decomposition of the hydroxyapatite matrix phase and increases the density of sintered material, thereby improving the mechanical properties of the composite. A sintered ceramic composite for implant prepared according to this method has mechanical properties at least three-times as high as those of hydroxyapatite, but has still insufficient mechanical properties to be applied to the load-bearing implant.

Currently, single phase zirconia (ZrO₂) and alumina (Al₂O₃) are primarily used as the load-bearing ceramic implant and a zirconia-alumina composite is being developed. Zirconia and alumina are widely used as high strength and high toughness ceramics. In addition, zirconia and alumina are known as bioinert materials which do not induce a toxic reaction when being inserted to a human body and are used as a patellar and a femoral head, which are load-bearing bones, among impaired bones. These bioinert ceramic materials cannot induce a chemical bonding with peripheral bones in a human body, thus, should be mechanically locked. However, when a load is applied to the implant after implantation, micro migration or movement of the implant occurs, which makes it difficult to secure initial immobility to peripheral tissues, and the implant is loosened due to trapping of fibrous tissues of a human body, resulting in a loss of function of the implant. Thus, it is urgently required to induce an active reaction with a human bone when the high strength bioinert ceramic is used as an implant.

### SUMMARY OF THE INVENTION

The present invention provides a sintered bioactive ceramic composite for implant, which has high strength and bioactivity, thereby securing initial immobility in a transplantation region.

The present invention also provides a method of preparing the sintered bioactive ceramic composite for implant.

According to an aspect of the present invention, there is provided a sintered bioactive ceramic composite for implant, including the zirconia-alumina nano-composite-powder and an apatite-related compound, wherein zirconia primary particles having a particle diameter of 10-50 nm and alumina primary particles having a particle diameter of 10-100 nm are sintered to form the nano-scale composite in a secondary particle state.

The apatite-related compound may be at least one compound selected from the group consisting of hydroxyapatite, carbonateapatite, fluoroapatite, oxyapatite, fluorohydroxyapatite, Sr-doped hydroxyapatite, Sr-doped carbonateapatite, Sr-doped fluoroapatite, Sr-doped oxyapatite, Sr-doped fluorohydroxyapatite, Mg-doped hydroxyapatite, Mg-doped carbonateapatite, Mg-doped fluoroapatite, Mg-doped oxyapatite, Mg-doped fluorohydroxyapatite, Si-doped hydroxyapatite, Si-doped carbonateapatite, Si-doped fluoroapatite, Si-doped oxyapatite, and Si-doped fluorohydroxyapatite.

The amount of the zirconia-alumina nano-composite-powder may be 50-99 vol %.

The amount of the apatite-related compound may be 1-50 vol %.

The content of zirconia in the zirconia-alumina nano-composite-powder may be 50-99.9 wt %.

According to another aspect of the present invention, there is provided a method of preparing the sintered bioactive ceramic composite for implant, including: preparing a zirconia-alumina nano-composite-powder; mixing the zirconia-alumina nano-composite-powder with an apatite-related compound; and sintering the resulting mixture.

50-99 vol % of the zirconia-alumina nano-composite-powder may be mixed with 1-50 vol % of the apatite-related compound.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view of a polymeric network structure in which zirconium ions and aluminium ions are trapped during preparing the zirconia-alumina nano-composite-powder according to an embodiment of the present invention;

FIG. 2 is a transmission electron microscope (TEM) image of a zirconia-alumina nano-composite-powder used in a sintered bioactive ceramic composite for implant according to Example of the present invention;

FIG. 3 is a schematic view of the zirconia-alumina nano-composite-powder;

FIG. 4 is a scanning electron microscope (SEM) image of the sintered bioactive ceramic composite for implant according to Example of the present invention;

FIG. 5A is a graph illustrating X-ray diffraction patterns of sintered bioactive ceramic composites for implant according to Example of the present invention and Comparative Example;

FIG. 5B is a graph illustrating phase decomposition of hydroxyapatite based on the x-ray diffraction analysis results;

FIG. 6 is a graph illustrating 4-point bending strength with respect to the amount of hydroxyapatite addition in the sintered bioactive ceramic composites for implant according to Example of the present invention and Comparative Example;

FIG. 7 is an SEM image of a sintered bioactive ceramic composite for implant according to Example of the present invention, on which osteoblastic cells are growing;

FIG. 8 is a graph illustrating the proliferation rate of osteoblast which is cultured on the sintered bioactive ceramic composite for implant according to Example of the present invention; and

FIG. 9 is a graph illustrating differentiation of osteoblast which is cultured on the sintered bioactive ceramic composite for implant according to Example of the present invention.

### DETAILED DESCRIPTION OF THE INVENTION

The present invention will now be described in greater detail.

A sintered bioactive ceramic composite implant according to an embodiment of the present invention includes the zirconia-alumina nano-composite-powder and an apatite-related compound, wherein zirconia primary particles having a particle diameter of 10-50 nm and alumina primary particle having a particle diameter of 10-100 nm are sintered to form the nano-scale composite in a secondary particle state.

Herein, the term “nano-composite-powder” refers to powder produced by nano-sintering at least two primary particles of nano-sized metal oxide to form a composite in a secondary particle state.

The apatite-related compound is in contact with the zirconia-alumina nano-composite-powder and improves bioactivity of the composite. The apatite-related compound may be represented by formula (1):
Ca₁₀(PO₄)₆Zₘ (1)

where Z is OH, CO₃, F, or Fₓ(OH)₁₋ₓ (0<x<1); and m is a number satisfying a valence, for example, 1 or 2. For example, hydroxyapatite may be represented by formula Ca₁₀(PO₄)₆(OH)₂. In particular, hydroxyapatite has bioactivity to form a strong chemical bond with peripheral bone tissues when it is transplanted in a body.

Examples of the apatite-related compound include hydroxyapatite, carbonateapatite, fluoroapatite, oxyapatite, fluorohydroxyapatite, Sr-doped hydroxyapatite, Sr-doped carbonateapatite, Sr-doped fluoroapatite, Sr-doped oxyapatite, Sr-doped fluorohydroxyapatite, Mg-doped hydroxyapatite, Mg-doped carbonateapatite, Mg-doped fluoroapatite, Mg-doped oxyapatite, Mg-doped fluorohydroxyapatite, Si-doped hydroxyapatite, Si-doped carbonateapatite, Si-doped fluoroapatite, Si-doped oxyapatite, Si-doped fluorohydroxyapatite, a mixture thereof, and a material which can be converted into apatite by sintering etc., i.e., a apatite precursor.

The zirconia-alumina nano-composite-powder is described in detail in Korean Patent Application No. 2004-80356 and Korean Patent Application No. 2005-0094526, which are filed by the applicant of the present application. That is, zirconia primary particles having a particle diameter of 10-50 nm and alumina primary particles having a particle diameter of 10-100 nm are sintered to form the nano-scale composite in a secondary particle state.

The zirconia-alumina nano-composite-powder may further include an oxide of at least one metal selected from the group consisting of yttrium, magnesium, calcium, cerium, niobium, scandium, neodymium, plutonium, praseodymium, samarium, europium, gadolinium, promethium, and erbium.

The zirconia-alumina nano-composite-powder can control decomposition, which occurs when it contacts with the apatite-related compound.

When the apatite-related compound and zirconia are mixed in a general method to form a sintered material, all the bioactive apatite-related compound is converted to bioresorbable tricalcium phosphate (Ca₃(PO₄)₂) due to an interfacial reaction between the apatite-related compound and zirconia, and thus a desired bioactivity cannot be expected, and the density of the sintered composite is reduced due to decomposition of the apatite-related compound and calcium oxide (CaO) which is a side product of reaction, induces phase transformation of zirconia, resulting in a reduction in mechanical property.

Meanwhile, the zirconia-alumina nano-composite-powder has a reduced surface area of zirconia particle to freely contact with the apatite-related compound, compared to a mixed zirconia/alumina powder which conventionally ball-milled at the same compositional ratio, thereby reducing decomposition of the apatite-related compound. In addition, the zirconia-alumina nano-composite-powder improves the strength of the sintered material by inhibiting growth of zirconia and alumina particles during sintering them.

The nano-composite-powder for zirconia-alumina sintered composite having the optimum strength may include 50-99.9 wt % of zirconia. Most preferably, the nano-composite-powder may include 80 wt % of zirconia and 20 wt % of alumina.

The amount of the zirconia-alumina nano-composite-powder in the sintered bioactive ceramic composite may be about 50-99 vol %, preferably about 60-80 vol %. The amount of the apatite-related compound in the composite may be about 1-50 vol %, preferably about 20-40 vol %. When the amount of the apatite-related compound is greater than 50 vol %, the strength of the sintered bioactive ceramic composite is reduced, which is not enough to be applied in load-bearing applications.

Due to a limited interfacial reaction between the apatite-related compound and the zirconia-alumina nano-composite-powder, the bioactive apatite-related compound and the bioresorbable tricalcium phosphate co-exists in proper amounts, and thus the bioresorbable tricalcium phosphate supplies a mineral ingredient of a new bone when osteoblast reacts with the apatite-related compound to produce the new bone. Thus, biphasic calcium phosphate (BCP) having bioactivity much better than that of a single phase apatite-related compound or a single phase tricalcium phosphate is produced.

In the sintered bioactive ceramic composite for implant, 0.1-60 parts by volume of the apatite-related compound may be converted into tricalcium phosphate.

A method of preparing the sintered bioactive ceramic composite for implant includes: preparing a zirconia-alumina nano-composite-powder; mixing the zirconia-alumina nano-composite-powder with an apatite-related compound; and sintering the resulting mixture.

A method of preparing the zirconia-alumina nano-composite-powder includes: mixing a mixed solution of polyhydric alcohol and carboxylic acid and a mixed solution of zirconium salt and aluminium salt; heating the mixture to 100-300° C. to form a polyester network structure in which zirconium ions and aluminum ions are trapped; and calcining the resultant at 400-1000° C.

The mixed solution of polyhydric alcohol and carboxylic acid forms the polyester network structure in presence of the mixed solution of zirconium salt and aluminum salt.

Examples of the polyhydric alcohol include ethylene glycol, propylene glycol, diethylene glycol, triethylene glycol, dipropylene glycol, hexylene glycol, butylene glycol, glycerol, hydroquinone(p-dioxybenzene), catechol(1,2-dihydroxybenzene), resorcinol (resorcin or 1,3-dioxybenzene), pyrogallol(1,2,3-trihydroxybenzene), 5-hydroxymethylresorcinol(3,5-dihydroxybenzyl alcohol), phloroglucinol(1,3,5-trihydroxy benzene), and dihydroxybiphenol, with ethylene glycol being most preferable.

Examples of carboxylic acid include citric acid, benzenetricarboxylic acid, cyclopentatetracarboxylic acid, adipic acid (1,4-butandicarboxylic acid), maleic acid (1,2-ethylenedicarboxylic acid), oxalic acid, succinic acid, tartaric acid (dioxysuccinic acid), mesaconic acid (methylfumaric acid), glutaric acid (n-pyrotartaric acid), malonic acid, glycolic acid, malic acid, lactic acid, gluconic acid, fumaric acid, phthalic acid (o-benzenedicarboxylic acid), isophthalic acid (m-benzenedicarboxylic acid), terephthalic acid, m-hydroxybenzoic acid, p-hydroxybenzoic acid, salicylic acid (o-hydroxybenzoic acid), itaconic acid (methylenesuccinic acid), citraconic acid, aconitic acid, galic acid, hydroxyethylethylenediaminetriacetic acid (HEDTA), ethyleneglycoltetraacetic acid (EGTA), ethylenediaminetetraacetic acid (EDTA), glutamic acid, aspartic acid, and ethylenediaminetetrapionic acid, with citric acid being most preferable.

The molar ratio of the polyhydric alcohol and the carboxylic acid may be 10:90 to 90:10. When the molar ratio of the polyhydric alcohol and the carboxylic acid is not within the range, the polyester network structure to trap metal ions is loose and the size of unit cell increases, which significantly reduces part to produce a nano-composite-powder, resulting in a reduction in yield.

The zirconium salt and the aluminum salt may be chloride, nitrate, or hydroxide.

The weight ratio of the zirconia-alumina powder obtained from the oxidation of Zr and Al ions to the mixed solution of polyhydric alcohol and carboxylic acid may be 10:1 to 10:999.9.

When the amount of the mixed solution of polyhydric alcohol and carboxylic acid is not within the range, a desired zirconia-alumina nano-composite-powder cannot be formed.

The mixed solution of zirconium salt and aluminum salt may further include at least one metal salt selected from the group consisting of yttrium, magnesium, calcium, cerium, niobium, scandium, neodymium, plutonium, praseodymium, samarium, europium, gadolinium, promethium, and erbium salts. The metals are included in zirconia to improve the physical property of zirconia. The metal salt may be present in the zirconia at a molar ratio thereof to zirconia of 0.0001-20:1 when it is transformed into an oxide.

Similarly to a network former and a network modifier in glass, the polyester network structure, which traps metal ions, may consist of a polymer network former and a metal cation network modifier. The metal ions as a network modifier are uniformly distributed in the polyester network in an atomic level. Generally, such a structure does not require diffusion over a broad region in a subsequent process of forming metal oxides and allows a stoichiometrically uniform single phase of metal oxide to be formed at a relatively low temperature.

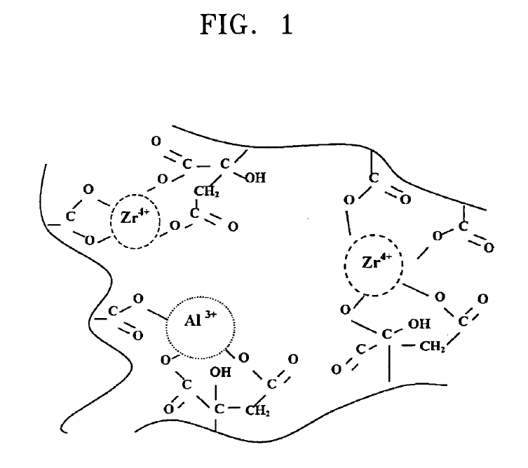

To form the zirconia-alumina nano-composite-powder according to an embodiment of the present invention, zirconium and aluminum are introduced into the polyester network as metal ions. Zirconium and aluminum ions are distributed in the polyester network as schematically illustrated in FIG. 1 and are oxidized by a subsequent thermal treatment to form primary particles of zirconia and alumina, which are sintered by successive heat treatment to form a nano-composite-powder having a secondary particle form. Since zirconia and alumina do not make solid-solution with each other, there is no potential of forming a single compound. Thus, zirconium and aluminum ions can closely contact with each other in the polymer network to form a nano-sized composite powder during a subsequent thermal treatment. Unlike the present invention, when polymer is not added to a metal ion solution, zirconium and aluminum salt particles are separately precipitated during thermally treating the metal salt solution and the precipitated metal salts are oxidized in the subsequent calcining process to give zirconia and alumina powders. The powder is composed of a mixture of micron-sized zirconia and alumina. The size of zirconia particles is 100-200 nm, whereas that of alumina particles is 500 nm or greater due to agglomeration of particles. The zirconia particles have tetragonal phase and the alumina particles are agglomerated from primary particles. Therefore, a microscopically uniformly mixed nano-composite-powder as in the present invention cannot be obtained.

The polymer precursor, i.e., the mixed solution of carboxylic acid and polyhydric alcohol is added to the mixed solution of zirconium salt and aluminum salt to form the zirconia-alumina nano-composite-powder. The zirconia-alumina nano-composite-powder begins to be formed and the alumina and zirconia agglomerate is reduced. All secondary particles are clusters having a size of 100-200 nm. The zirconia-alumina nanocomposite contains nanocrystalline zirconia having a size of about 10-50 nm. A polymer is added to a metal source (metal salt solution), which enables the polymer network to trap metal ions in a dissociated carboxyl group, thereby allowing the metal ions to remain adjacent to each other. The polymer is removed and the nano-composite-powder is formed by calcining the polymer network which traps zirconium ions and aluminum ions at a temperature of 400-1000° C. That is, aluminum and zirconium ions are uniformly dispersed/mixed in the polyester network structure at a molecular level and many zirconium ions act as nuclei for zirconium oxidation and subsequent oxide crystallite growth. The zirconia first grows into nano-sized particles and is dispersed and mixed with aluminum, which is oxidized at a relatively high temperature, at a molecular level to form a sintered composite powder of zirconia nanoparticles and alumina nanoparticles.

The resulting zirconia-alumina nano-composite-powder is mixed with an apatite-related compound.

Examples of the apatite-related compound include hydroxyapatite, carbonateapatite, fluoroapatite, oxyapatite, fluorohydroxyapatite, Sr-doped hydroxyapatite, Sr-doped carbonateapatite, Sr-doped fluoroapatite, Sr-doped oxyapatite, Sr-doped fluorohydroxyapatite, Mg-doped hydroxyapatite, Mg-doped carbonateapatite, Mg-doped fluoroapatite, Mg-doped oxyapatite, Mg-doped fluorohydroxyapatite, Si-doped hydroxyapatite, Si-doped carbonateapatite, Si-doped fluoroapatite, Si-doped oxyapatite, Si-doped fluorohydroxyapatite, and precursors thereof. The apatite precursor refers to a material which can be converted into apatite after sintering, for example, octacalcium phosphate, amorphous calcium phosphate, etc.

The resulting mixture is sintered to form a sintered ceramic composite.

That is, the mixture is hot pressed at a temperature of 1300-1400° C. under a pressure of 10-30 MPa and an Ar gas atmosphere for 1-3 hrs.

The present invention will now be described in greater detail with reference to the following examples. The following examples are for illustrative purposes only and are not intended to limit the scope of the invention.

### EXAMPLE

Preparation of Zirconia-Alumina Nano-Composite-Powder

Metal chloride was used as a cation source, and citric acid monohydrate and ethylene glycol were used as a polymer matrix.

AlCl₃.6H₂O, ZrCl₂O.8H₂O, YCl₃.6H₂O (available from Aldrich Chemical Co. Inc., Milwaukee, Wis., USA), C₆H₈O₇.H₂O (CAM), and ethylene glycol (C₂H₆O₂, EG) were used as starting materials. All materials except for YCl₃.6H₂O were obtained from Kanto Chemical Co Inc., Tokyo, Japan.

A stoichiometric mixture of Zr and Y sources (ZrO₂ doped with 3 mol % of Y₂O₃) and an Al source solution were used as starting materials. The polymer matrix was composed of CAM and EG at a molar ratio of 33:67 and the total amount of polymer was 90 parts by weight based on 10 parts by weight of the metal oxide and the weight ratio of alumina to zirconia was 0.25:1.

The metal sources were mixed with the CAM-EG solution. Then, the resulting mixture was heated at 130° C. to facilitate esterification between CAM and EG. As the solution was concentrated, it became very viscous, and turned from colorless to yellow, and then to brown in color. The resulting gel was dried, pulverized, and calcined at a temperature of 200-1000° C. The calcined powder was analyzed with an X-ray diffractometer (M18XHF, Mac Science, Yokohama, Japan). The powder was analyzed with TEM to inspect whether a nano-composite-powder was formed.

Preparation of Sintered Bioactive Ceramic Composite for Implant

60-90 vol % of the zirconia-alumina nano-composite-powder prepared above and 10-40 vol % of hydroxyapatite (Alfa Aesar Co., MA, USA) were mixed with each other, ball-milled, and then dried before being filtered through a sieve. The obtained powder was molded in a graphite mold under a low pressure, and then hot pressed under a pressure of 30 MPa at a temperature of 1400° C. for 1 hr under an Ar atmosphere.

### COMPARATIVE EXAMPLE

Preparation of Conventionally Ball-Milled Ceramic Composite of zirconia-alumina

Commercially available zirconia powder (TZ-3Y, Tosoh, Japan) and alumina powder (AKP 50, Sumitomo, Japan) with a particle diameter of 300 nm were conventionally ball-milled with each other, and then ball milled and dried before being filtered through a sieve. The obtained powder was hot pressed under the same conditions as in the above Example.

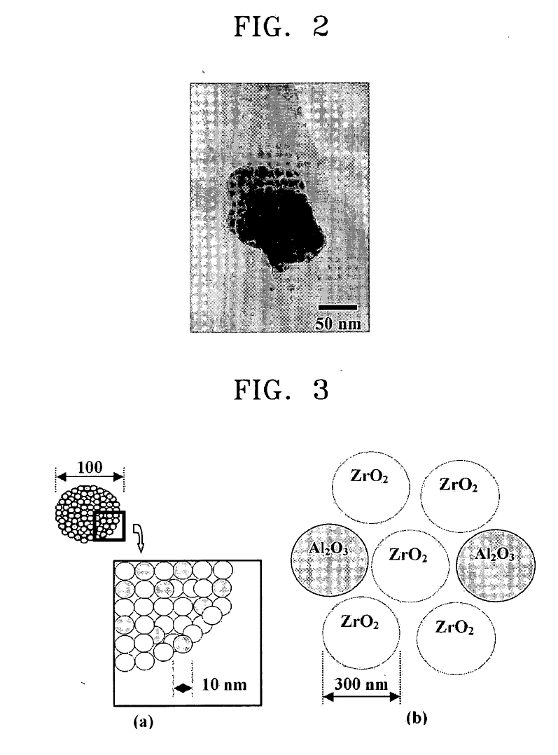

FIG. 2 is a TEM image of a zirconia-alumina nano-composite-powder used as a matrix phase in the sintered bioactive ceramic composite for implant according to Example of the present invention. Referring to FIG. 2, black zirconia particles with a particle diameter of about 10 nm were uniformly dispersed in the composite powder with a particle diameter of about 100 nm. Alumina particles appeared to be a matrix phase of the zirconia-alumina nano-composite-powder.

FIG. 3 schematically illustrated the zirconia-alumina nano-composite-powder and the mixed zirconia-alumina powder.

More specifically, referring to FIG. 3A, white zirconia and black alumina are uniformly distributed in the zirconia-alumina nano-composite-powder having a size of about 100 nm. Since only a small amount of nano-sized zirconia crystallites is present on the surface of the nano-composite-powder compared to the total amount of zirconia (80 wt %), when it forms the composite with hydroxyapatite, a contact area is reduced, which can inhibit decomposition of the matrix phase. Meanwhile, referring to FIG. 3B, in the case of Comparative Example, which used the powder prepared by simple mixing, a zirconia powder having a particle diameter of about 300 nm as a matrix phase can freely contact with hydroxyapatite so that a large amount of zirconia reacts with hydroxyapatite.

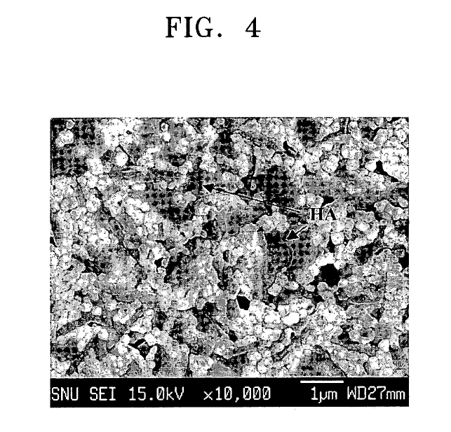

FIG. 4 is an SEM image of a sintered bioactive ceramic composite for implant according to Example of the present invention. This shows the microstructure of the composite consisting of the zirconia-alumina nano-composite-powder and hydroxyapatite. Light-colored small particles represent zirconia, heavy-colored long small particles represent alumina, and large round particles represent hydroxyapatite (HA). The number of micropores in the sintered material is small, which indicates high sintered density and uniform distribution of particles of each component.

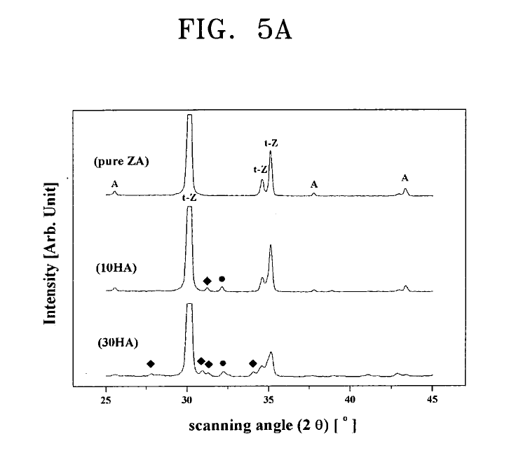

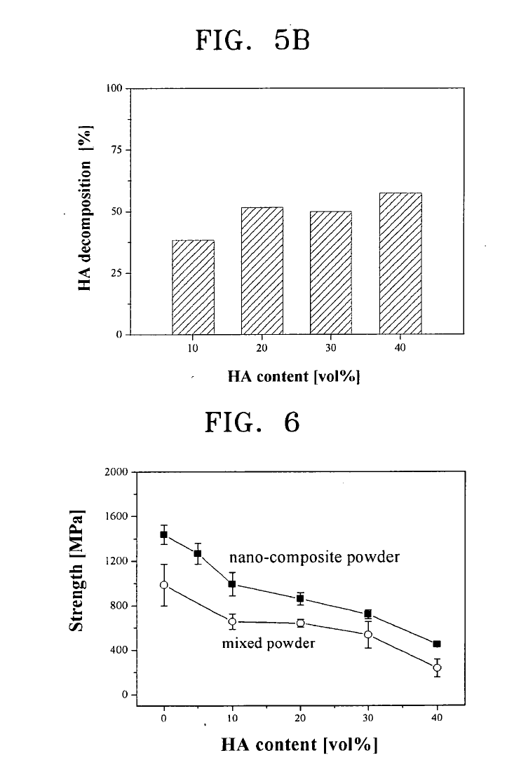

FIGS. 5A and 5B are graphs illustrating the X-ray diffraction patterns of sintered bioactive ceramic composite for implant according to Example of the present invention and Comparative Example and HA decomposition based on the results.

Specifically, referring to FIG. 5A, (pure ZA) represents the case in which a zirconia-alumina nano-composite-powder without containing hydroxyapatite was sintered; (10HA) represents a diffraction pattern of a composite containing 10 vol % of hydroxyapatite; and (30HA) represents a diffraction pattern of a composite containing 30 vol % of hydroxyapatite. In the zirconia-alumina nanocomposite without containing hydroxyapatite, an alumina (A) phase was detected together with tetragonal zirconia (t-Z) without monoclinic or cubic zirconia. When hydroxyapatite was added, diffraction peaks of hydroxyapatite (●) and tricalcium phosphate (♦) were observed together with the tetragonal zirconia and the alumina phase. As the amount of hydroxyapatite increased, the amount of tricalcium phosphate slightly increased, but the amount of the bioactive hydroxyapatite was similar to that of the bioresorbable tricalcium phosphate so that they were present as biphasic calcium phosphate (BCP), which had optimum bioactivity.

FIG. 5B schematically illustrated the amount of produced tricalcium phosphate with respect to the amount of hydroxyapatite in the bioactive ceramic composite (or the amount of decomposed hydroxyapatite) calculated based on the intensity of diffraction peak in the X-ray diffraction pattern. When 10 vol % of hydroxyapatite was added, approximately 37% of tricalcium phosphate was produced, and when 20-30 vol % of hydroxyapatite was added, approximately 50% of tricalcium phosphate was present in the composite. When 40 vol % of hydroxyapatite was added, approximately 60% of hydroxyapatite was converted into tricalcium phosphate.

FIG. 6 is a graph illustrating schematically 4-point bending strength of sintered bioactive ceramic composites for implant according to Example and Comparative Example.

Specifically, the mechanical strength of the sintered bioactive ceramic composite for implant which used the zirconia-alumina nano-composite-powder of Example of the present invention and that of the sintered composite which used the simple mixture of zirconia and alumina of Comparative Example were measured. Referring to FIG. 6, when hydroxyapatite was added, the strength of the sintered zirconia-alumina composite decreased. However, when the simple mixture of zirconia/alumina powders was used as a matrix phase, the strength of the sintered ceramic composite was significantly reduced compared to when the zirconia-alumina nano-composite-powder was used as the matrix phase. This matched the description regarding the schematic view illustrated in FIG. 3.

FIG. 7 is an SEM image of osteoblast which was cultured on the sintered bioactive ceramic composite for implant according to Example of the present invention. This is the result of an experiment conducted to assess the bioactivity of the sintered composite using osteoblast which generates human bone cells. As can be seen from the image, the osteoblast is growing on the composite.

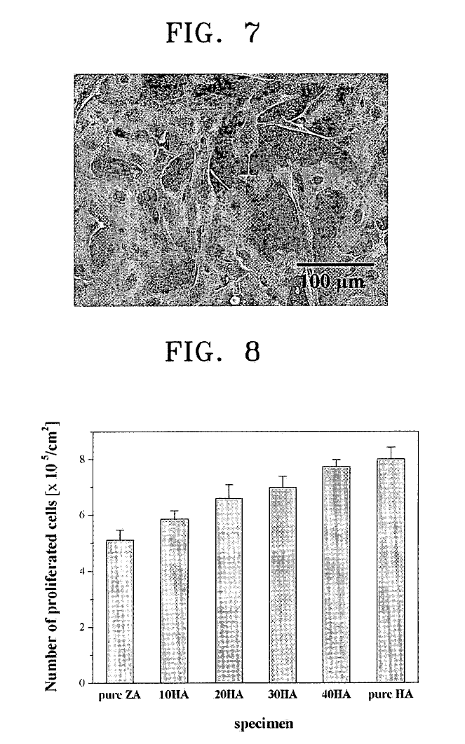

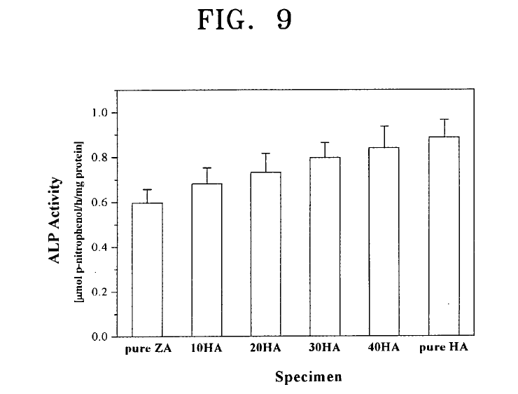

FIGS. 8 and 9 are schematic views for describing the improved bioactivity of the sintered bioactive ceramic composite for implant according to Example of the present invention.

FIG. 8 is a graph illustrating the proliferation rate of osteoblast which was cultured on the sintered bioactive ceramic composite for implant according to Example of the present invention.

Specifically, as the content of hydroxyapatite in the sintered bioactive ceramic composite increased, the proliferation of osteoblast was activated. This indicates that osteoblast exhibits higher proliferation in the bioactive ceramic than in the sintered zirconia-alumina composite which is bioinert ceramic. In particular, when 40 vol % of hydroxyapatite was added, cell proliferation pattern is almost close to single phase hydroxyapatite was shown.

FIG. 9 is a graph illustrating differentiation of osteoblast which was cultured on the sintered bioactive ceramic composite for implant according to Example of the present invention.

Specifically, as the amount of hydroxyapatite added to the composite increased, the differentiation of osteoblast increased, which shows the same increase pattern as the result of the proliferation rate of osteoblast.

As can be seen from the above results, the zirconia-alumina nano-composite-powder inhibited effectively the interfacial decomposition reaction of zirconia and the apatite-related compound to prevent significant decrease in the strength of the sintered ceramic composite and improve bioactivity. Accordingly, the zirconia-alumina nano-composite-powder can be used as the matrix phase of the load-bearing sintered bioactive ceramic composite for implant or as a secondary phase of non-load bearing sintered bioactive ceramic composite for implant. In addition, hydroxyapatite can be used as the secondary phase or matrix phase for improving the bioactivity of the sintered ceramic composite.

The compositional ratio of materials may vary depending on mechanical properties and biocompatibility required by part to which the sintered bioactive ceramic composite is applied.

The sintered ceramic composite according to an embodiment of the present invention contains the bioactive hydroxyapatite and bioresorbable tricalcium phosphate in proper amounts, and thus has good biocompatibility and can be applied to a load-bearing medical ceramic implant.

The zirconia-alumina nano-composite-powder is used as a matrix phase or a secondary phase to inhibit the growth of particles during a sintering process, thereby obtaining high strength and reducing the interfacial reaction of zirconia and the apatite-related compound during the sintering process. Thus, the zirconia-alumina nano-composite-powder can be effectively used when the improvement of mechanical properties is suppressed due to a serious interfacial reaction of the matrix phase and the secondary phase.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A sintered bioactive ceramic composite for implant, comprising a zirconia-alumina nano-composite-powder and an apatite-related compound, wherein zirconia primary particles having a particle diameter of 10-50 nm and alumina primary particles having a particle diameter of 10-100 nm are sintered to form the nano-scale composite in a secondary particle state.

2. The sintered bioactive ceramic composite for implant of claim 1, wherein the apatite-related compound is at least one compound selected from the group consisting of hydroxyapatite, carbonateapatite, fluoroapatite, oxyapatite, fluorohydroxyapatite, Sr-doped hydroxyapatite, Sr-doped carbonateapatite, Sr-doped fluoroapatite, Sr-doped oxyapatite, Sr-doped fluorohydroxyapatite, Mg-doped hydroxyapatite, Mg-doped carbonateapatite, Mg-doped fluoroapatite, Mg-doped oxyapatite, Mg-doped fluorohydroxyapatite, Si-doped hydroxyapatite, Si-doped carbonateapatite, Si-doped fluoroapatite, Si-doped oxyapatite, and Si-doped fluorohydroxyapatite.

3. The sintered bioactive ceramic composite for implant of claim 1, wherein an amount of the zirconia-alumina nano-composite-powder is 50-99 vol %.

4. The sintered bioactive ceramic composite for implant of claim 1, wherein an amount of the apatite-related compound is 1-50 vol %.

5. The sintered bioactive ceramic composite for implant of claim 4, wherein the amount of the apatite-related compound is 20-40 vol %.

6. The sintered bioactive ceramic composite for implant of claim 1, wherein a content of zirconia in the zirconia-alumina nano-composite-powder is 50-99.9 wt %.

7. The sintered bioactive ceramic composite for implant of claim 1, wherein the zirconia-alumina nano-composite-powder further comprises an oxide of at least one metal selected from the group consisting of yttrium, magnesium, calcium, cerium, niobium, scandium, neodymium, plutonium, praseodymium, samarium, europium, gadolinium, promethium, and erbium.

8. The sintered bioactive ceramic composite for implant of claim 1, wherein 0.1-60 parts by volume of the apatite-related compound is converted into tricalcium phosphate.

9. A method of preparing the sintered bioactive ceramic composite for implant, comprising:
preparing a zirconia-alumina nano-composite-powder;
mixing the zirconia-alumina nano-composite-powder with an apatite-related compound; and
sintering the resulting mixture.

10. The method of claim 9, wherein the preparing of a zirconia-alumina nano-composite-powder comprises:
mixing a mixed solution of polyhydric alcohol and carboxylic acid and a mixed solution of zirconium salt and aluminium salt;
heating the mixture to 100-300° C. to form a polyester network in which zirconium ions and aluminum ions are trapped; and
calcining the resultant at 400-1000° C.

11. The method of claim 9, wherein 50-99 vol % of the zirconia-alumina nano-composite-powder and 1-50 vol % of the apatite-related compound are mixed.

12. The method of claim 9, wherein the apatite-related compound is at least one compound selected from the group consisting of hydroxyapatite, carbonateapatite, fluoroapatite, oxyapatite, fluorohydroxyapatite, Sr-doped hydroxyapatite, Sr-doped carbonateapatite, Sr-doped fluoroapatite, Sr-doped oxyapatite, Sr-doped fluorohydroxyapatite, Mg-doped hydroxyapatite, Mg-doped carbonateapatite, Mg-doped fluoroapatite, Mg-doped oxyapatite, Mg-doped fluorohydroxyapatite, Si-doped hydroxyapatite, Si-doped carbonateapatite, Si-doped fluoroapatite, Si-doped oxyapatite, and Si-doped fluorohydroxyapatite.
